(19) United States
(12) Patent Application Publication
(10) Pub. No.: US 2004/0137025 A1
(43) Pub. Date: Jul. 15, 2004

(54) Water-in-oil emulsion cosmetic composition

(75) Inventors: Masaki Kosugi, Kashiwa-shi (JP); Junko Nakayama, Tokyo (JP)

Correspondence Address: GREENBLUM & BERNSTEIN, P.L.C., 1950 ROLAND CLARKE PLACE, RESTON, VA, 20191, US

(73) Assignee: KOSE Corporation, Tokyo (JP)

(21) Appl. No.: 10/703,517

(22) Filed: Nov. 10, 2003

(30) Foreign Application Priority Data
Nov. 14, 2002 (JP) 2002-330851

(51) Int. Cl. A61K 7/06; A61K 7/11; A61K 7/00

(52) U.S. Cl. 424/401; 424/70.12

(57) ABSTRACT

A novel water-in-oil emulsion cosmetic composition that comprises (a) an oily ingredient, (b) from 0.3 to 6 mass % of a dipolyhydroxystearic acid polyoxyethylene, (c) from 0.2 to 6 mass % of a particular class of silicone compound and (d) an aqueous ingredient is disclosed.

### BACKGROUND OF THE INVENTION

1. Field of the Invention

The present invention relates to a water-in-oil emulsion cosmetic composition that comprises an oily ingredient, a dipolyhydroxystearic acid polyoxyethylene, a particular class of silicone compound, and an aqueous ingredient, optionally further comprises a silicone oil, a branched-chain C₁₄₋₃₀ fatty acid ester, and an organic-modified clay mineral. More specifically, the invention relates to a water-in-oil emulsion cosmetic composition, comprising the above-mentioned ingredients, which provides a comfortable feeling when applied to the skin and excellent effect on the skin, and has good long-term stability.

2. Description of the Related Art

As compared with oil-in-water (O/W) emulsions for cosmetics, water-in-oil (W/O) emulsions for cosmetics have a high affinity for the skin, and have potent effects on the skin such that they prevent moisture transpiration and protect the skin from drying, as well as provide skin treatment effect. Therefore, they have conventionally been used for various cosmetics. However, most of W/O emulsions for cosmetics provide uncomfortable feelings such as heavy in spreading or sticky to the skin. To overcome these disadvantages, for example, addition of silicone oils or surfactants is disclosed in Japanese Patent Unexamined Publication (KOKAI) No. 2001-39819. Water-in-oil emulsion cosmetic compositions comprising the silicone oils and surfactants successfully provide improvement of feeling when applied to the skin, however, they fail to achieve high emollient effect. If a large amount of silicone oil, that is widely used in ordinary cosmetics to obtain high emollient effect, is added to W/O emulsions, problems often arise in phase separation, precipitation of crystals or the like due poor compatibility of the silicone-type oily material with ordinary raw materials.

For improvement of the stability of the W/O emulsions and feeling of use, i.e., light feeling in spreading or smooth feeling on the skin, Fragrance Journal, September1999, pp.83-86 discloses a means including application of a nonionic surfactant consisting of dipolyhydroxystearic acid polyoxyethylene.

### SUMMARY OF THE INVENTION

An object of the present invention is to provide a water-in-oil emulsion cosmetic composition which has a high compatibility to the skin, excellent feeling of use such as free from sticky feeling, and high emollient effect, as well as has a good long-term stability.

Under the above circumstances, the present inventors conducted various studies, and as a result, they found that, by using an oily ingredient, a dipolyhydroxystearic acid polyoxyethylene, a particular class of silicone compound, and an aqueous ingredient, a water-in-oil emulsion cosmetic composition was obtainable which had comfortable feel of use and potent effects on the skin as well as a good long-term stability. They also found that, by using a silicone oil, a branched-chain C₁₄₋₃₀ fatty acid ester, and further by using an organic-modified clay mineral, the cosmetic composition having synergistically improved feeling of use, effects on the skin, and long-term stability was obtainable. On the basis of these findings, the present invention was achieved.

In one aspect, the present invention provides a water-in-oil emulsion cosmetic composition comprising:

(a) an oily ingredient,

(b) from 0.3 to 6 mass % of a dipolyhydroxystearic acid polyoxyethylene,

(c) from 0.2 to 6 mass % of a silicone compound selected from the group consisting of:

R¹ₐR²_{b}R³_{c}SiO_{(4-a-b-c)}/2

wherein R¹ represents an organic group selected from the group consisting of a C₁₋₃₀ alkyl, an aryl, an aralkyl, a fluoroalkyl group, and an organic group of —CₘH₂ₘ—O—(C₂H₄O)_{d}(C₃H₆O)ₑR⁴, wherein plural R¹'s may be the same or different; R² represents a polyoxyalkylene group of —CₘH₂ₘ—O—(C₂H₄O)_{f}(C₃H₆O)_{g}R⁵; R³ represents an organosiloxane group of the following formula (2):

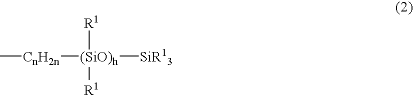

R⁴ represents a C₄₋₃₀ hydrocarbon group or an organic group of R⁶—(CO)—; R⁵ represents a hydrogen atom, a C₁₋₃₀ hydrocarbon group, or an organic group of R—(CO)—; R⁶ represents a C₁₋₃₀ hydrocarbon group; a, b, and c satisfy: 1.0≦a≦2.5, 0.001≦b≦1.5, and 0.001≦c≦1.5; d and e are integers of: 0≦d≦50 and 0≦e≦50; f and g are integers of: 2≦f≦200, 0≦g≦200, and 3≦(f+g)≦200; m is an integer of 0≦m≦15; h is an integer of 0≦h≦500; and n is an integer of 1≦n≦5, and

(d) an aqueous ingredient.

The present invention is also relates to the water-in-oil emulsion cosmetic composition wherein said composition comprises 6 to 50 mass % of the oily ingredient (a) which comprises a silicone oil as ingredient (e), and wherein a content of the ingredient (e) based on the ingredient (a) is 3 to 70 mass %.

The present invention further relates to the water-in-oil emulsion cosmetic composition, wherein the oily ingredient (a) comprises a branched-chain C₁₄₋₃₀ fatty acid ester as ingredient (f), and wherein a content of the ingredient (f) based on the ingredient (a) is 10 to 95 mass %.

The present invention also relates to the water-in-oil emulsion cosmetic further comprising (g) an organic-modified clay mineral.

### DESCRIPTION OF THE EMBODIMENTS

Embodiments of the present invention are described in detail below.

The oily ingredient (a) is a component that constitutes the oil phase of the water-in-oil emulsion cosmetic composition, and is used to obtain comfortable feeling such as rich and smooth spreading or softness, and effect on the skin such as an emollient effect or the like. Various oils generally used in ordinary cosmetics, which may be animal oils, vegetable oils or synthetic oils, and may be solid oils, semisolid oils, liquid oils or volatile oils, can be used as the ingredient (a). Examples of the ingredient (a) include hydrocarbons, fats, waxes, hardened oils, ester oils, fatty acids, higher alcohols, fluoro-oils, lanolin derivatives, oily gelling agents, oleophilic surfactants, oil-soluble UV absorbents. More specific examples of the ingredient (a) include hydrocarbons such as liquid paraffin, squalane, vaseline, polyisobutylene, polybutene, paraffin wax, ceresin wax, microcrystalline wax, montan wax, Fischer-Tropsch wax; fats such as olive oil, castor oil, mink oil, macadamia nut oil, Japan tallow; waxes such as bees wax, carnauba wax, candelilla wax, spermaceti wax, jojoba oil; esters such as octyldodecyl myristate, polyglyceryl diisostearate, diglyceryl triisostearate, glyceryl tribehanate, pentaerythritol rosinate, cholesterol fatty acid esters, di(cholesteryl-behenyl-octyldodecyl) N-lauroyl-L-glutamate; higher fatty acids such as stearic acid, lauric acid, myristic acid, behenic acid, isostearic acid, oleic acid, rosin acid; higher alcohols such as stearyl alcohol, cetyl alcohol, lauryl alcohol, oleyl alcohol, isostearyl alcohol, behenyl alcohol; fluoro-oils such as perfluoropolyether, perfluorodecane, perfluorooctane; lanolin and lanolin derivatives such as lanolin acetate, isopropyl lanolin fatty acid, lanolin alcohol; oily gelling agents such as dextrin fatty acid esters, sucrose fatty acid esters, starch fatty acid esters, 12-hydroxystearic acid, calcium stearate; and oil-soluble UV absorbents such as ethyl aminobenzoate, 2-ethylhexyl methoxysilicate, 4-tert-butyl-4′-methoxydibenzoylmethane, oxybenzone. These compounds may be used alone or two or more compounds may be used in combination.

The ingredient (b), dipolyhydroxystearic acid polyoxyethylene, is used to improve long-term stability of the water-in-oil emulsion cosmetic composition while to maintain the excellent feeling of use. The ingredient (b) may be obtained by reacting an acid chloride, which is prepared by halogenation of poly(12-hydroxystearic acid) with a halogenating agent such as thionyl chloride, with polyethylene glycol in the presence of an alkali. In this process, when a large amount of the acid chloride is used, a dipolyester is obtained, whilst a small amount of the acid chloride gives compound with a low acylation degree such as monoesters. A polyethylene glycol, when used for preparation of the ingredient (b) of the present invention, may preferably have a mean molecular weight of from 400 to 6000. Poly(12-hydroxystearic acid), which is the other constituting element of the ingredient (b), forms a hydrophobic moiety in the essential ingredient (b), and its degree of polymerization may preferably have a mean molecular weight of from 1000 to 3000. Some of the dipolyhydroxystearic acid polyoxyethylene compounds are available in the market, and such commercially-available products may be used in the present invention. Among the commercially-available products, a most preferred example includes ARLACEL P-135 (trade name, manufactured and sold by UNIQEMA). This product is a diester of poly(12-hydroxystearic acid) with polyethylene glycol (mean molecular weight, 1500), and has a mean molecular weight of about 5000. These dipolyhydroxystearic acid polyoxyethylenes may be used alone or two or more compounds may be used in combination. The ingredient (b) has an action of having the continuous phase exist stably in the emulsion system of the present invention.

The content of the ingredient (b) used in the present invention may be from 0.3 to 6% by mass (a percent value will be hereinafter indicated simply as “%”) on the basis of a total water-in-oil emulsion cosmetic composition. The content may preferably be from 1 to 3% from viewpoints of improvements of feeling of use and the long-term stability of the composition.

A silicone compound, as the ingredient (c) of the present invention, is used to improve undesired feeling of use such as heavy feeling in spreading or sticky feeling to skin, which are inherent to water-in-oil emulsion cosmetics, and to improve the long-term stability of the composition. The ingredient (c) is a compound represented by the aforementioned formula (I) which has excellent emulsifying property and achieves high emulsion stability.

The ingredient (c) is at least one silicone compound selected from the group consisting of:

R¹ₐR²_{b}R³_{c}SiO_{(4-a-b-c)}/2

wherein R¹ represents an organic group selected from the group consisting of a C₁₋₃₀ alkyl, an aryl, an aralkyl, a fluoroalkyl group, and an organic group of —CₘH₂ₘ—O—(C₂H₄O)_{d}(C₃H₆O)ₑR⁴, wherein plural R¹ s may be the same or different; R² represents a polyoxyalkylene group of —CₘH₂ₘ—O—(C₂H₄O)_{f}(C₃H₆O)_{g}R⁵; R³ represents an organosiloxane group of the following formula (2):

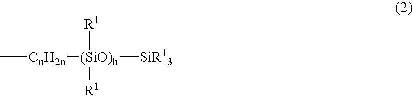

R⁴ represents a C₄₋₃₀ hydrocarbon group or an organic group of R⁶—(CO)—; R⁵ represents a hydrogen atom, a C₁₋₃₀ hydrocarbon group, or an organic group of R⁶—(CO)—; R⁶ represents a C₁₋₃₀ hydrocarbon group; a, b, and c satisfy: 1.0≦a≦2.5, 0.001≦b≦1.5, and 0.001≦c≦1.5; d and e are integers of: 0≦d≦50 and 0≦e≦50; f and g are integers of: 2≦f≦200, 0≦g≦200, and 3≦(f+g)≦200; m is an integer of 0≦m≦15; h is an integer of 0≦h≦500; and n is an integer of 1≦n≦5.

A mean molecular weight of the silicone compound of the above formula (1) is not specifically limited. The mean molecular weight may preferably be from 500 to 200,000, more preferably be from 1,000 to 100,000. A silicone compound having an HLB of from 2.5 to 6 is desirably used as an emulsifier or an emulsification aid in the water-in-oil emulsion cosmetic composition of the present invention. An example of commercially available products includes KF-6028 (trade name, manufactured and sold by Shin-Etsu Chemical Co., Ltd). The silicone compound as the ingredient (c) may be used alone or two or more compounds may be used in combination.

A content of the silicone compound used as the ingredient (c) may be from 0.2 to 6%, and preferably from 0.5 to 4% from viewpoints of improvements of the long term stability and feeling of use.

The aqueous ingredient (d) used in the present invention is a component that constitutes the aqueous phase of the water-in-oil emulsion cosmetic composition, and also is used to obtain excellent feeling of use such as a light feeling in spreading, reduced sticky feeling or fresh feeling on the skin, and to obtain effect on the skin such as moisturizing feeling. Examples of the ingredient (d) include purified water, polyalcohols such as glycerin, propylene glycol and butylene glycol, and ethanol. These ingredients may be used alone or two or more ingredients may be used in combination. In general, purified water, or a combination of purified water and a polyalcohol or ethanol is used.

By using the ingredients (a) to (d) according to the present invention, improvements of undesired feeling of use such as heavy feeling in spreading or sticky feeling to the skin can be achieved. Further, by using the aforementioned ingredients, the water-in-oil emulsion cosmetic composition also having improved long-term stability can be obtained.

By admixing the ingredient (e) in the oily gradient (a) used in the present invention, reduction of sticky feeling of use and synergistic improvement of fresh feeling of use can be achieved.

A chemical structure of the silicon oil as the ingredient (e) used in the present invention is not particularly limited. A linear, cyclic, or crosslinked silicone oil may be used. More specifically, examples include low-viscosity to high-viscosity silicone oils such as dimethylpolysiloxane, methylphenylpolysiloxane, methylhydrogenpolysiloxane, dimethylpolysiloxane/methylphenylpolysiloxane copolymer; cyclic silicone oils such as octamethylcyclotetrasiloxane, decamethylcyclopentasiloxane, dodecamethylcyclohexasiloxane, tetramethyltetrahydrogen-cyclotetrasiloxane, tetramethyltetraphneyl-cyclotetrasiloxane; higher alkoxy-modified silicones such as stearoxysilicone; higher fatty acid-modified silicones, amino-modified silicones and fluorine-modified silicones. These silicone oils may be used alone or two or more silicon oils may be used in combination. Among them, preferred examples include cyclic silicone oils such as octamethylcyclotetrasiloxane and decamethylcyclopentasiloxane from a viewpoint that fresh feeling is readily obtained.

A content of the oily ingredient (a) that comprises the silicone oil (e) may be from 6 to 50% on the basis of the total water-in-oil emulsion cosmetic composition of the present invention.

A content of the silicone oil (e) on the basis of the ingredient (a) may be from 3 to 70%, and preferably from 6 to 50% from viewpoints of reduction of sticky feeling to the skin and improvement of fresh feeling of use.

By admixing the ingredient (e) in the oily ingredient (a), an excellent W/O emulsion cosmetic composition can be obtained which has synergistic improvements in feeling of use, such as reduced heavy feeling in spreading, reduced sticky feeling to the skin and improved fresh feeling, while maintains a good long-term stability.

Further, by admixing ingredient (f), a branched-chain C₁₄₋₃₀ fatty acid ester in the oily gradient (a) used in the present invention, further improvements of compatibility to the skin and emollient effect on the skin can be achieved.

Specifically, examples of the branched-chain C₁₄₋₃₀ fatty acid ester as the ingredient (f) used in the present invention include isopropyl palmitate, isopropyl myristate, isopropyl stearate, isobutyl stearate, 2-ethylhexyl stearate, isopropyl isostearate, butyl isostearate, decyl isostearate, lauryl isostearate, isodecyl isodecanoate, isodecyl isononanoate, isotridecyl isononanoate, isononyl isononanoate, neopentyl glycol di-2-ethylhexanoate, propylene glycol di-2-ethylhexanoate, and glyceryl tri-2-ethylhexanoate. Among them, preferred examples include isotridecyl isononanoate, isononyl isononanoate, neopentyl glycol di-2-ethylhexanoate and glyceryl tri-2-ethylhexanoate each having 2 or more branched-chain structures from a viewpoint that they are readily mixable in cosmetic compositions and can achieve good long-term stability of the composition. If desired, these branched-chain C₁₄₋₃₀ fatty acid esters may be used alone or two or more esters may be used in combination.

A content of the branched-chain C₁₄₋₃₀ fatty acid ester as the ingredient (f) may be from 10 to 95%, and preferably from 20 to 80% from viewpoints of improvements of compatibility to the skin and emollient effect.

By admixing the ingredient (f) in the oily ingredient (a) used in the present invention, an excellent W/O emulsion cosmetic composition can be obtained which has synergistic improvements in feeling of use such as compatibility to the skin and in effect on the skin such as an emollient effect, while maintains the long-term stability.

The organic-modified clay mineral as the ingredient (g) according to the present invention may be used to further improve the stability of the W/O emulsion cosmetic composition. As the organic-modified clay minerals being the ingredient (g) of the present invention, those produced by addition of cationic organic amine compounds to natural or synthetic smectite clay minerals such as bentonite through a cation-exchange reaction. Examples of commercial available products of such organic-modified clay minerals include those launched by US National Lead Co in the market as tradenames of Benton 38 and Benton 27. It is also possible to use other water-swellable clay minerals such as vermiculite after being treat with cationic compounds so as to be organic-modified clay mineral having any modification degree for application in the present invention. It is also possible to add the water-swellable clay mineral and a cationic compound separately to the composition to form the organic-modified clay mineral in the system of the water-in-oil emulsion composition of the present invention. These organic-modified clay minerals as the ingredient (g) may be used alone or two or more may be used in combination.

A content of the organic-modified clay mineral as the ingredient (g) added in the composition of the present invention may be from 0.01 to 10% based on the total composition, and may preferably be from 0.1 to 5% from viewpoints of the long-term stability and feeling of use.

By using the ingredients (a) to (g), an excellent water-in-oil emulsion cosmetic composition can be obtained which has synergistic improvement in feeling of use and in effect on the skin, while maintains a long-term stability can be obtained.

In addition to the above-mentioned ingredients, ingredients used in ordinary cosmetics, for example, surfactants other than the ingredients (a) and (b), as well as electrolytes, polymer substances, moisturizing ingredients, aesthetic ingredients, microbicides, preservatives, inorganic powders, organic powders, antioxidants, dye, fragrances and the like may be used for the water-in-oil emulsion cosmetic composition of the invention so far that they do not affect the advantageous effect of the invention.

The water-in-oil emulsion cosmetic composition of the invention may be prepared by any ordinary preparing processes. For example, the composition may be obtained by a dispersive emulsification process in which an aqueous phase is added to an oily phase for emulsification.

Examples of practical applications of the water-in-oil emulsion cosmetic composition of the present invention include, for example, skin care cosmetics such as milky lotion, face cream, massage cream, hand cream, body cream, suntan cream; makeup cosmetics such as foundation, makeup cream, control cream; hair care cosmetics such as hair milk, hair cream. Examples of method for use of the composition include application by hand or use of infiltrated nonwoven fabric.

### EXAMPLES

The invention is described in more detail with reference to the following Examples, which, however, are not intended to restrict the scope of the invention.

Examples 1 to 8 and Comparative Examples 1 to 6, Water-in-Oil Emulsion Cosmetic Composition:

Water-in-oil emulsion cosmetic compositions shown in Table 1 below were prepared and evaluated their long-term stability and feeling of use.

TABLE 1

| | | Example | | | | | | | |
|---|---|---|---|---|---|---|---|---|---|
| | Ingredient | 1 | 2 | 3 | 4 | 5 | 6 | 7 | 8 |
| 1 | Aracel P-135 (note 1) | 0.3 | 3 | 6 | 1.5 | 1.5 | 1.5 | 1.5 | 1.5 |
| 2 | KF-6028 (note 2) | 1.5 | 1.5 | 1.5 | 0.2 | 6 | 1.5 | 1.5 | 1.5 |
| 3 | decamethyl- | — | — | — | — | — | 3 | 3 | 3 |
| | cyclopentasiloxane | | | | | | | | |
| 4 | Glyceryl tri-2- | — | — | — | — | — | — | 5 | 5 |
| | ethylhexanoate | | | | | | | | |
| 5 | pentaerythritol | 13 | 13 | 13 | 13 | 13 | 10 | 5 | 5 |
| | tetra-2- | | | | | | | | |
| | ehtylhexanoate | | | | | | | | |
| 6 | Benton 27 (note 3) | — | — | — | — | — | — | — | 1 |
| 7 | citric acid | 0.05 | 0.05 | 0.05 | 0.05 | 0.05 | 0.05 | 0.05 | 0.05 |
| 8 | 1,3-butylene glycol | 10 | 10 | 10 | 10 | 10 | 10 | 10 | 10 |
| 9 | Glycerin | 5 | 5 | 5 | 5 | 5 | 5 | 5 | 5 |
| 10 | pure water | balance | balance | balance | Balance | balance | balance | balance | balance |
| 11 | methyl p-oxybenzoate | q.s. | q.s. | q.s. | q.s. | q.s. | q.s. | q.s. | q.s. |
| 12 | dye (Red No. 106) | q.s. | q.s. | q.s. | q.s. | q.s. | q.s. | q.s. | q.s. |
| 13 | Fragrance | q.s. | q.s. | q.s. | q.s. | q.s. | q.s. | q.s. | q.s. |
| Tested Items and Results | | | | | | | | | |
| long-term stability | | ◯ | ◯ | ◯ | ◯ | ◯ | ◯ | ◯ | ⊚ |
| Feel | compatibility | ◯ | ◯ | ◯ | ◯ | ◯ | ◯ | ⊚ | ⊚ |
| of | to the skin | | | | | | | | |
| use | non-stickiness | ◯ | ◯ | ◯ | ◯ | ◯ | ⊚ | ⊚ | ⊚ |
| | emollient effect | ◯ | ◯ | ◯ | ◯ | ◯ | ◯ | ⊚ | ⊚ |

| | | Comparative Example | | | | | |
|---|---|---|---|---|---|---|---|
| | Ingredient | 1 | 2 | 3 | 4 | 5 | 6 |
| 1 | Aracel P-135 (note 1) | — | 3 | 3 | 3 | 3 | — |
| 2 | KF-6028 (note 2) | 3 | — | — | — | — | 3 |
| 3 | decamethyl- | 3 | 3 | — | 3 | 3 | 3 |
| | cyclopentasiloxane | | | | | | |
| 4 | Glyceryl tri-2- | 5 | 5 | 5 | — | 5 | 5 |
| | ethylhexanoate | | | | | | |
| 5 | pentaerythritol | 5 | 5 | 8 | 10 | 5 | 5 |
| | tetra-2- | | | | | | |
| | ehtylhexanoate | | | | | | |
| 6 | Benton 27 (note 3) | 1 | 1 | 1 | 1 | — | — |
| 7 | citric acid | 0.05 | 0.05 | 0.05 | 0.05 | 0.05 | 0.05 |
| 8 | 1,3-butylene glycol | 10 | 10 | 10 | 10 | 10 | 10 |
| 9 | Glycerin | 5 | 5 | 5 | 5 | 5 | 5 |
| 10 | pure water | balance | balance | balance | balance | balance | balance |
| 11 | methyl p-oxybenzoate | q.s. | q.s. | q.s. | q.s. | q.s. | q.s. |
| 12 | dye (Red No. 106) | q.s. | q.s. | q.s. | q.s. | q.s. | q.s. |
| 13 | Fragrance | q.s. | q.s. | q.s. | q.s. | q.s. | q.s. |
| Tested Items and Results | | | | | | | |
| long-term stability | | X | X | X | X | X | X |
| Feel | compatibility | Δ | Δ | Δ | X | Δ | Δ |
| of | to the skin | | | | | | |
| use | non-stickiness | Δ | Δ | X | Δ | Δ | Δ |
| | emollient effect | ◯ | ◯ | ◯ | X | ◯ | ◯ |

Note 1: produced by UNIQEMA Co.
Note 2: produced by Shin-etsu Chemical Industry Co.
Note 3: Produced by National Lead Co.
q.s. sufficient quantity

(Production Process)

A: Ingredients 1 to 6 and 11 were mixed and dissolved homogenously at 70° C.

B: Ingredients 7 to 10 and 12 were mixed homogenously at 70° C.

C: The mixture B was added to the mixture A, emulsified and cooled, and ingredient 13 was added to the mixture to obtain a water-in-oil emulsion cosmetic composition.

(Evaluation Method)

1. Long-term stability:

The water-in-oil emulsion cosmetic compositions in Table 1 were left stand in a thermostat at 5° C. or 50° C. for 1 month. Changes in appearance of the compositions were observed and evaluated according to the following criteria:

⊚: No change was found.

◯: Slight changes were found, but the changes were not problematic.

Δ: The tendency of separation was observed in the sample at either 50° C. or 5° C.

X: Apparent separation was observed in the sample at either 50° C. or 5° C. or in both of the samples at both 50(C or 5(C.

2. Feeling when applied to the skin:

Sensory evaluations of tests of the water-in-oil emulsion cosmetic compositions shown in Table 1 were carried out by ten women panelists as for the following items. The panelist's evaluations were based on the standards mentioned below.

Tested Items:

Whether or not the sample had a good compatibility to the skin: yes (+) or not (−).

Whether or not the sample was sticky during and after its application to skin: yes (−) or not (+).

Whether or not the sample has a high emollient effect after its application to skin: yes (+) or not (−).

The criteria for evaluation are as follows:

⊚: At least 8 panelists gave (+).

◯: 6 or 7 panelists gave (+).

Δ: 3 to 5 panelists gave (+).

X: 2 or less panelists gave (+).

The results obtained according to the above-mentioned evaluation methods are shown in Table 1.

As is understandable from the results, Examples 1 to 8 according to the present invention gave good long-term stability and good feeling when applied to the skin. Whilst, among Comparative Examples 1 to 6, no sample gave satisfactory results in all of the evaluation items.

### Example 9

### W/O Solid Foundation

| | |
|---|---|
| (Ingredient) | (%) |

| | | |
|---|---|---|
| 1. | Organic-modified smectite (note 1) | 1.0 |
| 2. | Ethanol | 5.0 |
| 3. | 1,3-butylene glycol | 5.0 |
| 4. | Sodium citrate | 0.5 |
| 5. | Fragrance | q.s. |
| 6. | Pure water | balance |
| 7. | Preservative | q.s. |
| 8. | Barium sulfate-coated mica titanium | 1.5 |
| 9. | Silicone-coated titanium oxide | 7.5 |
| 10. | Silicone-coated yellow iron oxide | 0.4 |
| 11. | Silicone-coated red iron oxide | 0.07 |
| 12. | Silicone-coated black iron oxide | 0.01 |
| 13. | KF-6028 (note 2) | 2.0 |
| 14. | Aracel P-135 (note 3) | 1.0 |
| 15. | Octamethylcyclotetrasiloxane | 7.0 |
| 16. | Isononyl isononanoate | 10.0 |
| 17. | Sucrose fatty acid ester | 1.0 |
| 18. | Isotridecyl isononanoate | 3.0 |
| 19. | Candelilla wax | 4.0 |
| 20. | Microcrystalline wax | 2.0 |
| 21. | Tocopherol sulfate | 0.05 |

(Note 1)
produced by National Lead Co.
(Note 2)
produced by Shin-etsu Chemical Industry Co.
(Note 3)
produced by UNIQEMA Co.

q.s. sufficient quantity

(Production Process)

A: Ingredients 1 to 7 were mixed and dissolved homogenously at 70° C.

B: Ingredients 8 to 21 were mixed homogenously at 70° C.

C: The mixture A was added to the mixture B and emulsified.

D: The emulsion was poured into a container at 70° C., and after the filling, the emulsion was left stand for cooling.

The water in oil solid foundation of Example 9 according to the present invention thus obtained above had good compatibility to the skin during use and high moisturizing effect on the skin, and was free from sticky feeling to the skin and gave high emollient feeling after use. The foundation also had good makeup effect on the skin as well a good long-term stability, which revealed that the foundation had excellent properties.

### Example 10

### Massage Cream

| | |
|---|---|
| (Ingredient) | (%) |

| | | |
|---|---|---|
| 1. | Polyvinyl alcohol | 1.5 |
| 2. | Methyl cellulose | 0.02 |
| 3. | Propylene glycol | 5.0 |
| 4. | Dipropylene glycol | 10.0 |
| 5. | Glycerin | 10.0 |
| 6. | Polyethylene glycol 400 | 2.0 |
| 7. | Sodium lactate | 0.5 |
| 8. | Sodium pyroglutamate | 1.3 |
| 9. | Preservative | q.s. |
| 10. | Pure water | balance |
| 11. | ABIL EM90 (note 1) | 2.0 |
| 12. | KF-6028 (note 2) | 0.7 |
| 13. | Aracel P-135 (note 3) | 2.0 |
| 14. | Decamethylcyclopentasiloxane | 2.0 |
| 15. | Isotridecyl isononanoate | 5.0 |
| 16. | Glyceryl tri-2-ethylhexanoate | 3.0 |
| 17. | Organic-modified bentonite (note 4) | 1.0 |
| 18. | Fragrance | q.s. |

(Note 1)
produced by Gold Schmitt Co.
(Note 2)
produced by Shin-etsu Chemical Industry Co.
(Note 3)
produced by UNIQEMA Co.
(Note 4)
produced by National Lead Co.

q.s. sufficient quantity

(Production Process)

A: Ingredients 1 to 10 were mixed and dissolved homogenously at room temperature.

B: Ingredients 11 to 18 were mixed homogenously at room temperature.

C: The mixture A was added to the mixture B and emulsified, and then cooled.

The massage cream of Example 10 according to the present invention obtained in the manner as above had good compatibility to the skin during use and high moisturizing effect on the skin, and was free from sticky feeling to the skin. The cream also gave high emollient feeling after use and had a good long-term stability.

### Example 11

### Eye lotion

| | |
|---|---|
| (Ingredient) | (%) |

| | | |
|---|---|---|
| 1. | KF-6028 (note 1) | 2.0 |
| 2. | Aracel P-135 (note 2) | 2.0 |
| 3. | Organic-modified bentonite (note 3) | 1.0 |
| 4. | Glyceryl tri-2-ethylhexanoate | 6.0 |
| 5. | Decamethylcyclopentasiloxane | 10.0 |
| 6. | Dimethylpolysiloxane | 3.0 |
| 7. | Silicons-processed mica titanium | 2.0 |
| 8. | Tocopherol acetate | 0.1 |
| 9. | Propylene glycol | 5.0 |
| 10. | Dipropylene glycol | 10.0 |
| 11. | Glycerin | 3.0 |
| 12. | Polyoxyethylene (10 E.O.) methyl glucoside | 3.0 |
| 13. | Magnesium ascorbate phosphate | 2.0 |
| 14. | Preservative | q.s. |
| 15. | Fragrance | q.s. |
| 16. | Pure water | balance |

(Note 1)
produced by Shin-etsu Chemical Industry Co.
(Note 2)
produced by UNIQEMA Co.
(Note 3)
produced by National Lead Co.

q.s. sufficient quantity

(Production Process)

A: Ingredients 1 to 7 were mixed and dissolved homogenously at room temperature.

B: Ingredients 8 to 14 were mixed homogenously at room temperature.

C: The mixture B was added to the mixture A and emulsified.

The eye lotion of Example 11 according to the present invention obtained in the manner as above had good compatibility to the skin during use, had high moisturizing effect on the skin, and was free from sticky feeling to the skin. The lotion also gave high tautness and emollient feeling after use, and had a good long-term stability, which revealed that the lotion had excellent properties.

Industrial Applicability

The present invention provides the water in oil cosmetic emulsion composition, which comprises an oily ingredient, a dipolyhydroxystearic acid polyoxyethylene, a specific silicone compound, and an aqueous ingredient, thereby achieves good feeling of use and effect on the skin, as well as long-term stability. The composition optionally comprises a silicone oil, a branched-chain C₁₄₋₃₀ fatty acid ester, or an organic-modified clay mineral, thereby the W/O cosmetic emulsion composition further excellent in the feeling of use, effect on the skin, and long-term stability can be obtained.

The present disclosure relates to subject matter contained in Japanese Patent Application No. 2002-330851, filed on Nov. 14, 2002, the contents of which is herein expressly incorporated by reference in its entirety.

What is claimed is:

1. A water-in-oil emulsion cosmetic composition comprising:
(a) an oily ingredient,
(b) from 0.3 to 6 mass % of a dipolyhydroxystearic acid polyoxyethylene,
(c) from 0.2 to 6 mass % of a silicone compound selected from the group consisting of:
R¹ₐR²_{b}R³_{c}SiO_{(4-a-b-c)}/2
wherein R¹ represents an organic group selected from the group consisting of a C₁₋₃₀ alkyl, an aryl, an aralkyl, a fluoroalkyl group, and an organic group of —CₘH₂ₘ—O—(C₂H₄O)_{d}(C₃H₆O)ₑR⁴, wherein plural R¹ s may be the same or different; R represents a polyoxyalkylene group of —CₘH₂ₘ—O—(C₂H₄O)_{f}(C₃H₆O)_{g}R⁵; R³ represents an organosiloxane group of the following formula (2): 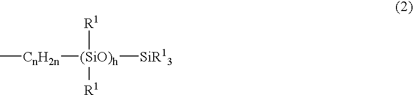
R⁴ represents a C₄₋₃₀ hydrocarbon group or an organic group of R⁶—(CO)—; R⁵ represents a hydrogen atom, a C₁₋₃₀ hydrocarbon group, or an organic group of R⁶—(CO)—; R⁶ represents a C₁₋₃₀ hydrocarbon group; a, b, and c satisfy: 1.0≦a≦2.5, 0.001≦b≦1.5, and 0.001≦c≦1.5; d and e are integers of: 0≦d≦50 and 0≦e≦50; f and g are integers of: 2≦f≦200, 0≦g≦200, and 3≦(f+g)≦200; m is an integer of 0≦m≦15; h is an integer of 0≦h≦500; and n is an integer of 1≦n≦5, and
(d) an aqueous ingredient.

2. The water-in-oil emulsion cosmetic composition of claim 1, comprising from 6 to 50 mass % of the oily ingredient (a) which comprises a silicone oil as ingredient (e).

3. The water-in-oil emulsion cosmetic composition of claim 2, wherein the content of the silicone oil (e) is from 3 to 70 mass % based on the oily ingredient (a).

4. The water-in-oil emulsion cosmetic composition of claim 1, wherein the oily ingredient (a) comprises (f) a branched-chain C₁₄₋₃₀ fatty acid ester.

5. The water-in-oil emulsion cosmetic composition of claim 4, wherein the content of the branched-chain C₁₄₋₃₀ fatty acid ester (f) is from 10 to 95 mass % based on the oily ingredient (a).

6. The water-in-oil emulsion cosmetic composition of claim 1, further comprising (g) an organic-modified clay mineral.

7. The water-in-oil emulsion cosmetic composition of claim 1, wherein the at least one dipolyhydroxystearic acid polyoxyethylene (b) is ARLACEL P-135 (trade name, manufactured and sold by UNIQEMA).

8. The water-in-oil emulsion cosmetic composition of claim 1, wherein the silicone compound (c) is selected from the group consisting of the compounds denoted by the formula (1) having a mean molecular weight of from 500 to 200,000.

9. The water-in-oil emulsion cosmetic composition of claim 8, wherein the silicone compound (c) is KF-6028 (trade name, manufactured and sold by Shin-Etsu Chemical Co., Ltd).

10. The water-in-oil emulsion cosmetic composition of claim 2, wherein the silicone oil (e) is selected from the group consisting of cyclic silicone oils.

11. The water-in-oil emulsion cosmetic composition of claim 4, wherein the branched-chain C₁₄₋₃₀ fatty acid ester (f) is selected from the group consisting of branched-chain C₁₄-30 fatty acid esters having two or more branched-chain structures.
